(19) United States
(12) Patent Application Publication
(10) Pub. No.: US 2009/0299248 A1
(43) Pub. Date: Dec. 3, 2009

(54) PORTABLE TRACTION DEVICE FOR TRACTION THERAPY AND METHODS OF USE THEREOF

(76) Inventor: Byung Kwon Cha, Brooklyn Park, MN (US)

Correspondence Address: DICKE, BILLIG & CZAJA, FIFTH STREET TOWERS, 100 SOUTH FIFTH STREET, SUITE 2250, MINNEAPOLIS, MN, 55402, US

(21) Appl. No.: 12/129,922

(22) Filed: May 30, 2008

(51) Int. Cl. A61F 5/00

(52) U.S. Cl. 602/36; 602/32

(57) ABSTRACT

A portable traction therapy device including a traction spring, a belt, and a traction control unit. The traction control unit interconnects the spring and the belt, and includes a locking mechanism and a switch assembly. The locking mechanism selectively locks and releases the belt relative to a housing of the control unit, with the switch assembly operating to dictate a state of the locking mechanism.

### BACKGROUND

The present disclosure relates to traction therapy devices. More particularly, it relates to a portable traction therapy device adapted to permit a user to quickly and easily apply traction therapy on an intermittent basis.

Traction devices are useful tools in providing therapeutic benefit to patients that have some type of disease or injury. Traction devices are used in traction therapy by providing a force that stretches or pulls on an affected joint or joints. These devices provide traction force to various body parts including the neck, wrist and hip joint. The traction device is usually attached on one end to a fixed stable object and the other is attached to the patient.

While traction therapy devices can assume a wide variety of forms. In many professional caregiver settings, for example, available traction therapy devices can be highly complex, relative large machines. Patients oftentimes desire to receive traction therapy at other, non-caregiver locations, such as at home. Obviously, the traction therapy machines used by many caregivers are not conducive to in-home use, due to size and cost. To better address the needs of patients, then, several portable, relative inexpensive traction therapy devices have been devised. U.S. Pat. No. 7,144,380 describes one such traction therapy device including a spring and a ratchet pulley control mechanism that can be mounted to commonly available structures, such as a door. While viable, the traction device of U.S. Pat. No. 7,144,380, as well as similar traction therapy devices, may be less-than-optimal. For example, available, portable traction therapy devices do not readily facilitate application of traction therapy on an intermittent basis. More particularly, existing portable traction therapy devices apply a relatively constant tension to the patient; in the event the patient wishes to temporarily reduce or cease the applied tension, he or she is effectively required to remove the corresponding harness. When re-application of tension is desired, the patient must then re-assemble the harness (and possibly other components of the traction therapy device) to the body part in question and re-initiate the device. This is a time-consuming process, and does not result in conventional, intermittent therapy.

In light of the above, a need exists for a portable traction therapy device affording a user the ability to easily provide intermittent therapy.

### BRIEF DESCRIPTION OF THE DRAWINGS

FIG. 1 is a perspective view of a traction therapy device in accordance with the present disclosure;

FIG. 2 illustrates use of the traction therapy device of FIG. 1;

FIG. 3A is a cross-sectional view of the traction therapy device of FIG. 1 in an un-locked state;

FIG. 3B is a side view of a portion of the traction therapy device in the state of FIG. 3A;

FIG. 3C is a cross-sectional view of the traction therapy device of FIG. 1 in a locked state;

FIG. 3D is a side view of a portion of the traction therapy device in the state of FIG. 3C;

FIG. 4A is an exploded view of a portion of the traction therapy device of FIG. 1, including a switch assembly component of a traction control unit;

FIG. 4B is a side view, with portions shown in phantom, of the switch assembly of FIG. 4A;

FIGS. 5A-5E are side views illustrating operation of the switch assembly of FIG. 4A;

FIGS. 6A-6E are side, cross-sectional views illustrating operation of the traction therapy device of FIG. 1 in applying intermittent traction therapy; and

FIGS. 7A and 7B are cross-sectional views illustration transitioning of the traction therapy device of FIG. 1 between differing applied tension levels.

### SUMMARY

Aspects of the present disclosure relate to a portable traction therapy device useful in applying intermittent traction therapy. The device includes a belt, a traction spring and a traction control unit. The belt has first and second ends, adapted to be connected, for example, to a harness and a handle, respectively. The traction spring is adapted for mounting to an external structure, such as a wall or a door. The traction control unit interconnects the belt and the traction spring. More particularly, the traction control unit includes a switch assembly that is configured to selectively lock and release the belt relative to the tension spring in response to a user-applied force. In some embodiments, the switch assembly includes a push button-type mechanism operable in response to a user applied force at the second end of the belt, with the switch assembly serving to lock and release the belt relative to a pulley about which the belt is fed. Regardless, during use, the traction spring is mounted to an external surface, and the belt mount to the patient (e.g., via the optional harness). By operating to lock and release the belt, the traction control unit facilitates intermittent therapy, where desired, by the user intermittently pulling on the belt (e.g., via the optional handle) while at all times connected to the belt (e.g., the optional harness need not be removed) with minimal effort.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top”, “bottom”, “front”, “back”, “leading”, “trailing” etc., is used with reference to the orientation of the Figure(s) being described. Because components of embodiments can be positioned in an number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

It is to be understood that the features of the various exemplary embodiments described herein may be combined with each other, unless specifically noted otherwise.

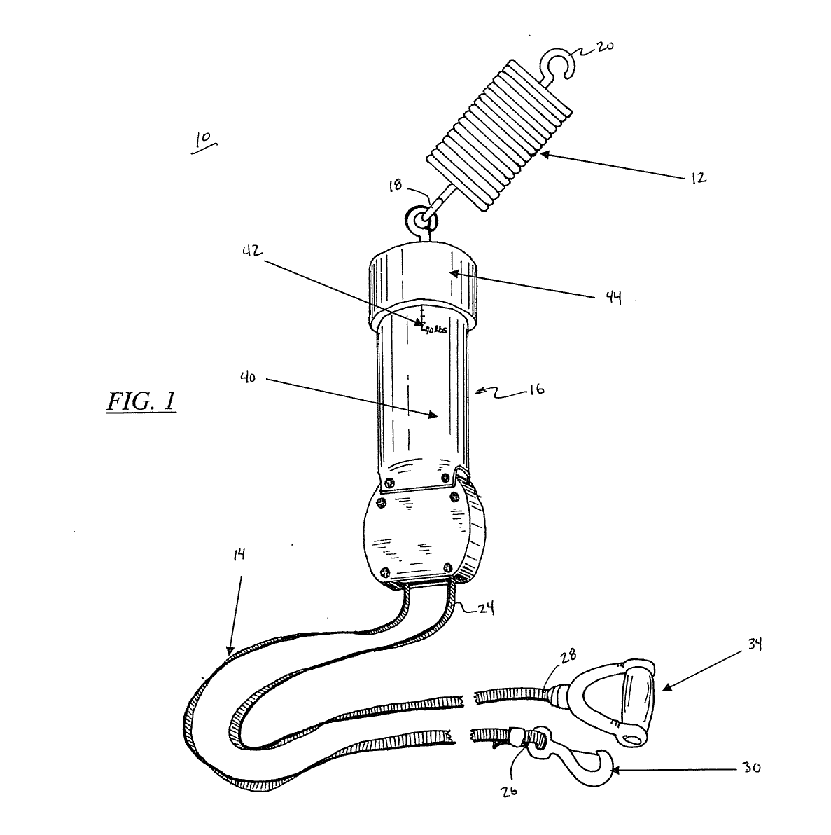

One embodiment of a portable traction therapy device 10 is shown in FIG. 1 and includes a traction spring 12, a belt 14, and a traction control unit 16. Details on the various components are provided below. In general terms, however, the traction control unit 16 interconnects the traction spring 12 and the belt 14, transferring forces between the components 12, 14. In this regard, the traction control unit 16 operates to selectively lock and release the belt 14 in response to a user-applied force.

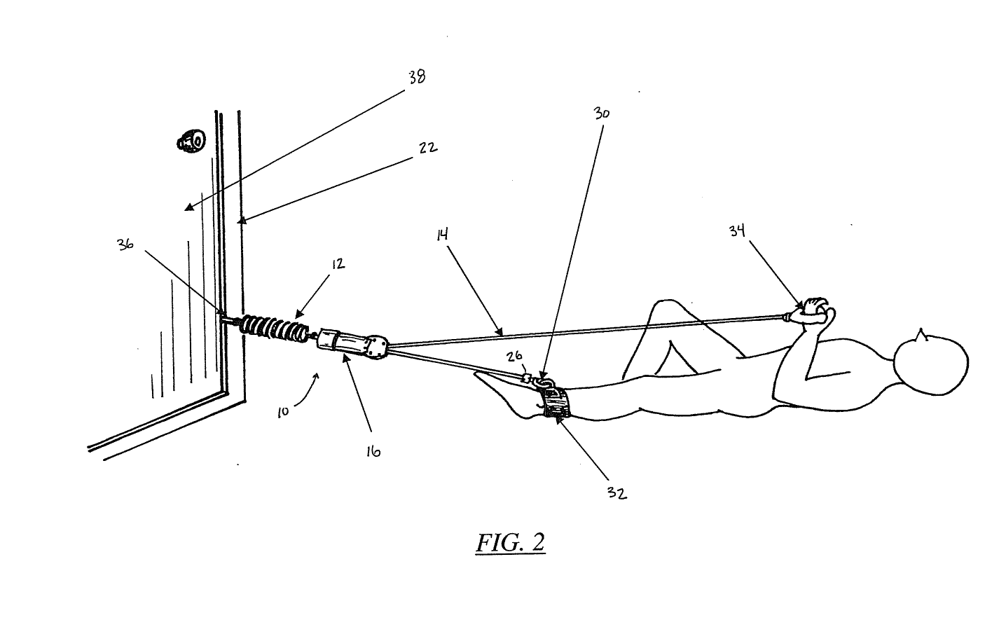

The traction spring 12 can assume a variety of forms (e.g., a helical coil extension spring), and is generally adapted for mounting to an external structure and connection to the traction control unit 16. For example, a first end 18 can form a tab or similar structure appropriate for interfacing with the traction control unit 16 as described below. Similarly, a second end 20 can form a hook or similar shape for releasable mounting to a door frame 22 as shown in FIG. 2. Alternatively, the traction device 10 can include additional devices or mechanisms (not shown) that facilitate connection of the second end 20 to the door frame 22 or other, desired external structure.

Returning to FIG. 1, the belt 14 can similarly assume a variety of forms and in some embodiments is a flexible, substantially inelastic reinforced fabric material strip that forms a corrugated-like outer surface 24. Regardless, the belt 14 terminates at opposing, first and second ends 26, 28. The ends 26, 28 form or are attached to components adapted for interfacing therewith by a user. For example, a clasp 30 or similar device can be assembled to the first end 26. The clasp 30 is adapted for selective mounting to a harness 32, one example of which is shown in FIG. 2 as adapted for securement about a patient's appendage. The harness 32 can assume a variety of other forms desired by users, such as a head-mounted harness. Even further, the harness 32 can be permanently connected to the first end 26, such that the clasp 30 is optional.

The second end 28 is, in some embodiments, assembled to a handle 34. Once again, the handle 34 can assume a variety of forms, and is generally provided for grasping by a user in applying a actuation force to the traction control unit 16 as described below. In other embodiments, the handle 34 can be omitted.

Regardless of an exact form, the belt 14 is threaded or otherwise fed through the traction control unit 16, with the ends 26, 28 available for operation by the user as shown in FIG. 2. As a point of reference, FIG. 2 illustrates just one of many examples of how the device 10 can be applied for traction therapy. In one embodiment were the traction device 10 is employed in traction therapy of a hip joint, the clasp is attached to the harness 32 that is wrapped around the user's ankle. The traction spring 12 is then hooked onto a door anchor strap 36 that is installed to the door frame 22 adjacent a door 38. Now the user pulls the handle 34 to initiate application of a traction force onto the user's leg via operation of the traction control unit 16.

With the above, general understanding of the therapy device 10 in mind, and with initial reference to FIG. 1, the traction control unit 16 includes a housing 40 maintaining various other components as described below. In general terms, however, the housing 40 can display a traction force indicating scale 42. Further, an end cap 44 may be included to adjust the applied traction force as described below, with the traction force indicating scale 42 providing an accurate readout of the applied traction force.

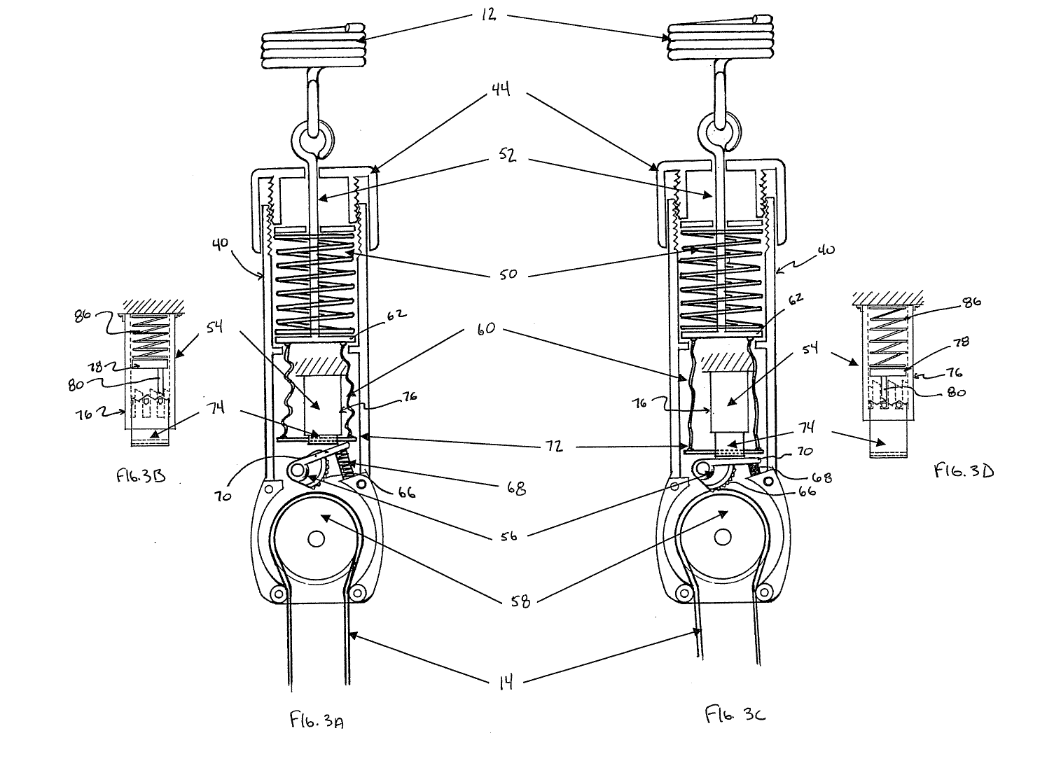

Additional components of the traction control unit 16 are shown in FIGS. 3A and 3B, and include a tension spring (e.g., a coil compression spring or other resistance device) 50, a piston 52, a switch assembly 54, a ratchet 56, and a pulley 58. In some embodiments, one or more wires 60 (e.g., steel wires) attach the piston 52 to the switch assembly 54. The wire(s) 60 may be joined by a variety of means including welding or crimping. It is contemplated that other materials could be substituted for the wires 60, including other metals and fibers. Regardless, the components 50-58 are assembled to, and maintained by, the housing 40 as shown, with the end cap 44 being threadably secured to the housing 40.

The piston 52 is slidably assembled within the housing 40, and is configured for mounting to the traction spring 12. The tension spring 50 is disposed between a head 62 of the piston 52 and the end cap 44, and translates a force (or resistance to force) applied to piston 52 (by the traction spring 12) onto the housing 40 (via the end cap 44). Thus, by adjusting a distance of the end cap 44 relative to the head 62, an effective force applied by the tension spring 50 to the housing 40 (and ultimately the belt 14 as described below) is similarly adjusted. Finally, the head 62 is connected to the wire(s) 60 as described above.

The pulley 58 is sized to receive the belt 14 as shown, and is rotatably mounted to the housing 40. The ratchet 56 is pivotably assembled to the housing 40 proximate the pulley 58. More particularly, the ratchet 56 forms an engagement surface 66 adapted to engage the belt 14 when the ratchet 56 is pivoted from the position of FIG. 3A to the position of FIG. 3C. A biasing member 68 (e.g., a spring) is mounted between an arm 70 of the ratchet 56 and surface of the housing 40, and biases the ratchet 56 to the released state of FIG. 3A in which the belt 14 freely moves with movement of the pulley 58. Alternatively, structure(s) or mechanism(s) differing from the ratchet 56 can be employed to effectuate locking and release of the belt 14.

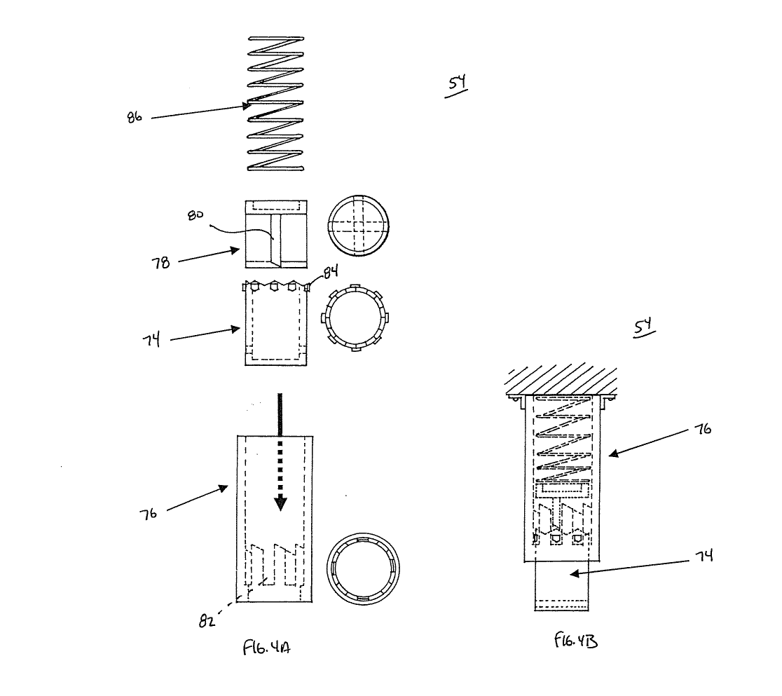

With additional reference to FIG. 4A, the switch assembly 54 is akin to a ball-point pen push button assembly, and generally includes a pin 72 mounted to a push button cup 74 that is slidably coupled to a case 76. The pin 72 is connected to the wire(s) 60 as described above. An indexing body 78 is rotatably maintained within the case 76, and includes a finger 80 adapted to selectively interface with grooves 82 of the case 76 and protrusions 84 of the push button 74. Finally, a switch spring 86 biases the indexing body 78 into selective engagement with the push button 74 and the case 76.

In the arrangement of FIGS. 3A and 3B, the switch assembly 54 is in the released state. More particularly, the indexing body 78 is positioned to draw the push button 74 into the case 76, thus maneuvering the push button 74 upwardly (relative to the orientation of FIGS. 3A and 3B). In this orientation, the ratchet spring 68 pushes up on the arm 70 of the ratchet 56, such that the ratchet 56 releases the belt 14 (e.g., the belt 14 is not locked against the pulley 58). Conversely, with a subsequent “push” of the push button 74, the indexing body 78 forces (via the switch spring 86) the push button 74 outwardly from the case 76 and into engagement with the arm 70. This, in turn, causes the ratchet 56 to lock the belt 14 against the pulley 58 as shown in FIGS. 3C and 3D. This same relationship of the switch assembly 54 is reflected in FIG. 4B.

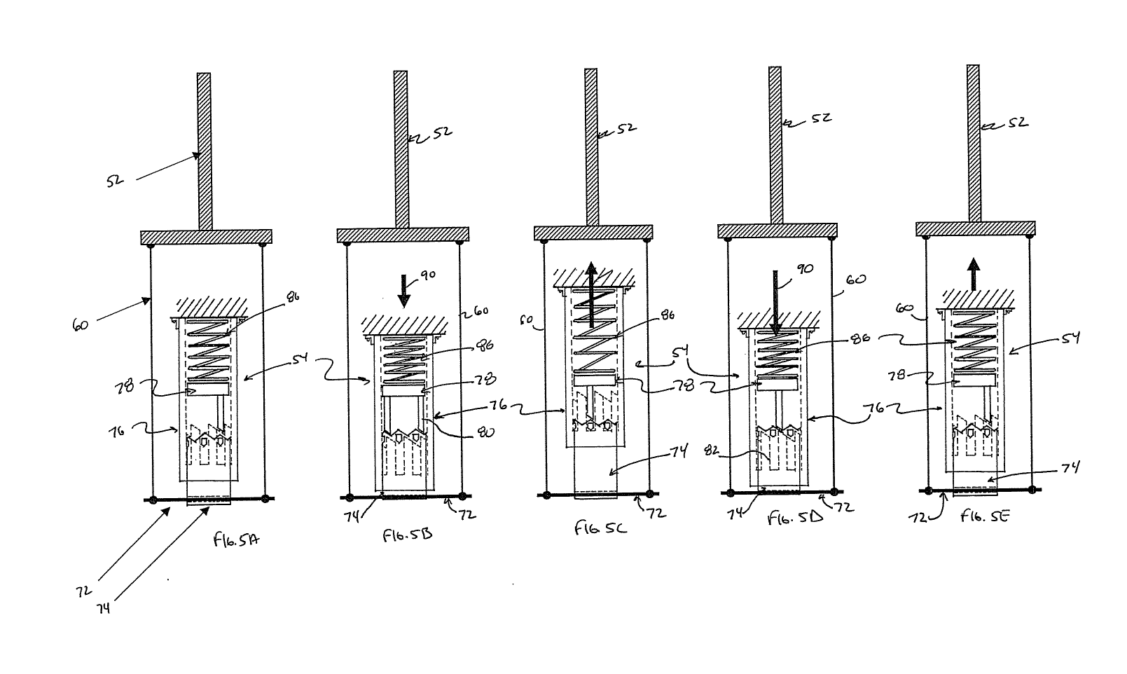

FIGS. 5A-5E illustrate the switch assembly 54 according to some embodiments cycling through states of being disengaged, to engaged and back to disengaged. The traction control unit 16 (FIG. 1) moves a whole with movement of the switch assembly 54 because the switch assembly 54 is fixed to the housing 40 (FIG. 1). In FIG. 5A all parts are at rest and are not being acted upon. In FIG. 5B, a force 90 is applied to the piston 52. Specifically, the piston 52 is pulled in a direction away from the switch assembly 54. The force 90 then pulls the switch assembly 54 by means of the wire(s) 60 attached to the pin 72 that otherwise runs through the push button cup 74. Movement of the push button 74 into the case 76 forces the switch spring 86 to compress and the indexing body 78 to rotate.

When the force 90 is released, the piston 52 moves in the opposite direction, relaxing the wire(s) 60. As the wire(s) 60 relax, the push button cup 74 moves out of the case 76. This allows the finger 80 to enter one of the grooves 82. The switch spring 84 then pushes the push button 74 out of the case 76, such that the switch assembly 54 is now engaged as seen in FIG. 5C.

To reverse the process, the same force 90 is again applied to the piston 52 as see in FIG. 5D. The only change in the process is that when the push button 74 is pushed into the case 76, the finger 80 of the indexing body 78 is rotated away from the grooves 82, thereby preventing the switch spring 84 from pushing the push button cup 74 out of the case 76. In this position, the switch assembly 54 is disengaged as seen in FIG. 5E. This operation of the switch assembly 54 can thus be similar to that of a ball point pen.

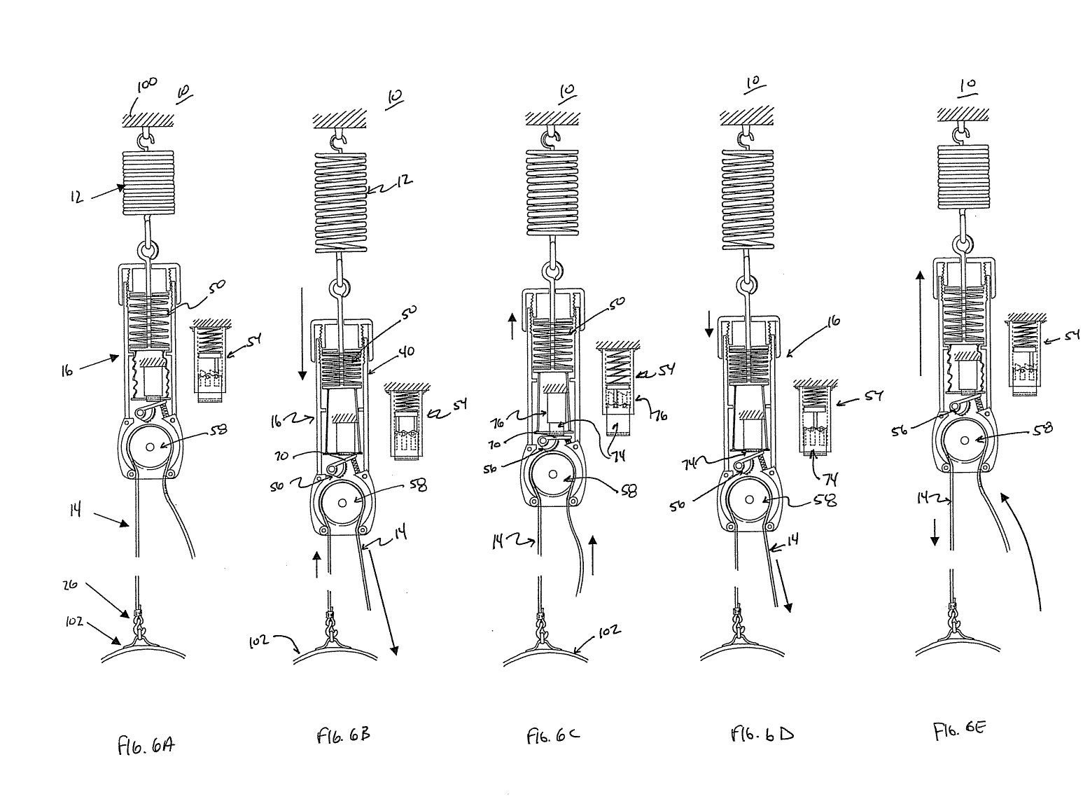

FIGS. 6A-6E illustrate use of the traction therapy device 10 cycling through intermittent application of traction therapy via repeatedly locking and releasing the belt 14 relative to the traction control unit 16. As a point of reference, FIGS. 6A-6E show the switch assembly 54 in operation as previously described in relation to FIGS. 5A-5E. The traction spring 12 is mounted to a structure 100, and the first end 26 of the belt 14 is connected to a harness 102. The harness 102, in turn, is mounted to the user/patient (not shown). While the second end 28 (FIG. 1) of the belt 14 is omitted from the views of FIGS. 6A-6E, it will be understood that the second end 28 is connected to the handle 34 (FIG. 1) as described above. Further, the switch assembly 54 is shown separately in each of FIGS. 6A-6E for purposes of clarity. Finally, FIG. 6A reflects the traction control unit 16 in a released or unlocked state, whereby the belt 14 is freely movable with rotation of the pulley 58.

Following assembly of the traction therapy device 10 as described above, in some embodiments when the user pulls down (as reflected in FIG. 6B) on the handle 34, the harness 102 remains stationary (as it is worn by the user). The belt 14 thus applies a downward force onto the traction control unit 16 via attachment of the pulley 58 to the housing 40. The traction control unit 16 thus moves downwardly (reflected by an arrow in FIG. 6B), with the traction spring 12, in turn, forced to extend. Conversely, the tension spring 50 is gradually compressed, increasing its resistance to compression. As the pulling force reaches a preset level of resistance force dictated by the switch assembly 54, the pulling force causes the piston 52 to be pulled in a direction opposite the force applied by the user and the wire(s) 60 become taught, thereby causing the switch assembly 54 to change states. In some constructions, the switch assembly 54 emits an audible “click” noise, prompting the user to cease application of the pulling force on the handle 34. In response, the traction control unit 16 moves slightly upward (via biasing of the tension spring 50). As the traction control unit 16 moves, the push button cup 74 projects from the case 76 and engages the arm 70 of the ratchet 56, rotating the ratchet 56 into contact with the belt 14 and locking the belt 14 against the pulley 58 as shown in FIG. 6C. In the locked state, the belt 14 is then locked into place preventing the relief of the traction force applied to the harness 102 (and thus the user).

To release the so-applied traction force, the user simply again pulls on the handle 34, causing the traction control unit 16 to move downwardly as in FIG. 6D. This action, in turn, cases the switch assembly 54 to press the push button 74, resulting in an audible “click” noise. In response to this prompt, the user decreases or ceases the pulling force on the handle 34, and the ratchet 56 is biased away from the belt 14 as shown in FIG. 6E. For an intermittent traction therapy, these same steps are repeated as desired by the user in selectively applying and releasing a an applied traction force.

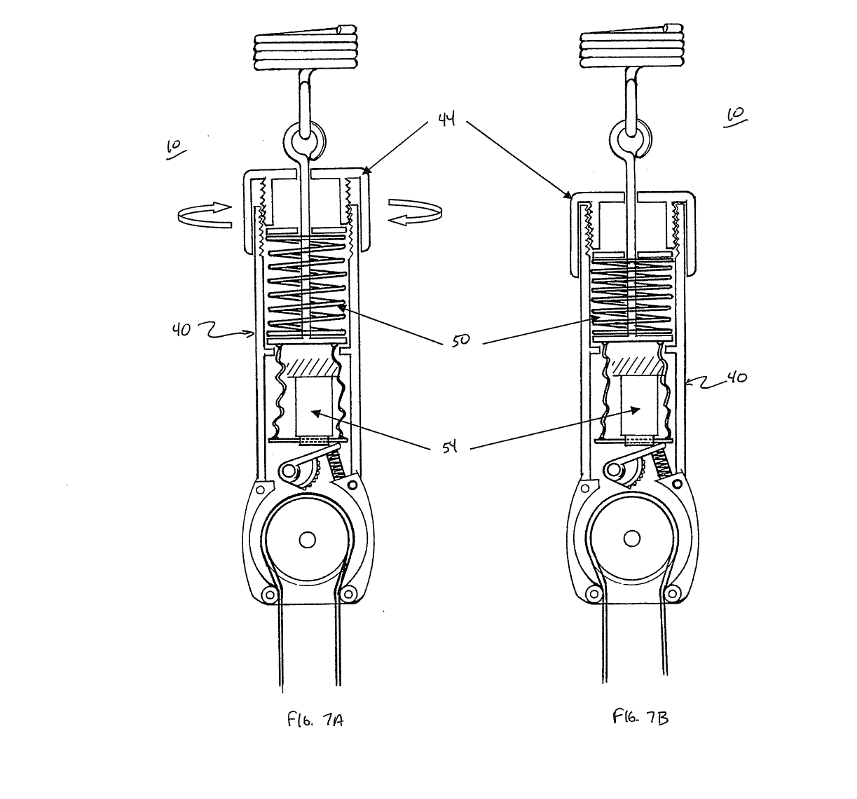

As mentioned above, in some embodiments the traction therapy device 10 is configured to allow a user to adjust the level of applied traction-type force. For example, and with reference to FIGS. 7A and 7B, the end cap 44 is threadably mounted to the housing 40. In order to increase the level of applied traction force, a user simply rotates the end cap 44 clock-wise as shown in FIG. 7A. As the end cap 44 is turned clockwise, the end cap 44 threads further onto the housing 40 and therefore compresses the tension spring 50 as shown in FIG. 7B resulting in more tension being delivered to by the device 10. Notably, this same action may cause the switch assembly 54 to require a greater force to effectuate operation thereof. As shown in FIG. 1, the indicating scale 42 correlates a position of the end cap 44 relative to the housing 40 with an applied traction force.

Although the present disclosure has been described with reference to preferred embodiments, workers skilled in the art will recognize that changes can be made in form and detail without departing from the spirit and scope of the present disclosure.

What is claimed is:

1. A portable traction therapy device comprising:
a traction spring adapted for mounting to an external structure;
a belt defining opposing, first and second ends;
a harness attached to the first end of the belt;
a handle attached to the second end of the belt; and
a traction control unit interconnecting the traction spring and the belt and including a locking mechanism and a switch assembly, the locking mechanism adapted to selectively lock and release the belt in response operation of the switch assembly by a user-applied force.

2. The traction therapy device of claim 1, wherein the traction control unit further includes:
a housing;
a pulley rotatably assembled to the housing, wherein the belt is fed about a the pulley; and
a ratchet pivotably mounted within the housing proximate the pulley for selectively locking the belt against the pulley in response to operation of the switch assembly.

3. The traction therapy device of claim 2, wherein the traction control unit further includes:
a piston having a head slidably disposed within the housing and connectable to the compression spring; and
a connector interconnecting the head with the switch assembly.

4. The traction therapy device of claim 3, wherein the traction control unit further includes:
a tension spring disposed between the head and an internal surface of the housing;
wherein the tension spring transfers a force from the piston onto the housing, and the housing transfers a force from the tension spring onto the pulley.

5. The traction therapy device of claim 4, wherein the traction control unit further includes:
an end cap threadably secured to the housing and bearing against an end of the tension spring opposite the head.

6. The traction therapy device of claim 2, wherein the traction control unit further includes:
a biasing disposed between an arm of the ratchet and a surface of the housing for biasing the ratchet away from the pulley.

7. The traction therapy device of claim 1, wherein the switch assembly includes:
a case;
an indexing body rotatably and slidably mounted within the case;
a biasing member bearing against the indexing body; and
a push button cup slidably captured to the case such that a portion of the cup is selectively extendable from the case.

8. The traction therapy device of claim 7, wherein the indexing body includes a finger and is transitionable between a first position in which the finger engages the cup and second position in which the finger is free of the cup.

9. The traction therapy device of claim 7, wherein the control unit further includes a piston connectable to the traction spring, and a pin mounted to the cup and connected to the piston.

10. The traction therapy device of claim 9, wherein the control unit further includes at least one flexible, inextensible wire interconnecting the pin and the piston.

11. The traction therapy device of claim 1, wherein the belt includes a corrugated outer surface.

12. The traction therapy device of claim 11, wherein the traction control unit includes a ratchet forming a toothed surface for selectively engaging the belt.

13. A method of providing self-traction therapy comprising:
providing a portable traction therapy device including:
a traction spring,
a belt defining opposing, first and second ends,
a harness attached to the first end of the belt,
a handle attached to the second end of the belt,
a traction control unit interconnecting the traction spring and the belt and including a locking mechanism and a switch assembly, the locking mechanism adapted to selectively lock and release the belt in response to operation of the switch assembly;
mounting the traction spring to an external structure;
attaching the harness to a part of a user's body; and
operating the switch assembly to a deployed position in response to a first user applied force to the handle to lock the belt relative to the traction control unit;
wherein the traction therapy device applies traction tension to the user with the locking mechanism in the locked state.

14. The method of claim 13, further comprising:
operating the switch assembly to a retracted position in response to a second user applied force to the handle to release the belt relative to the traction control unit;
wherein the traction therapy device releases the applied traction tension to the user with the locking mechanism in the released state.

15. The method of claim 14, further comprising:
intermittently applying the first and second user forces in performing an intermittent traction therapy.

16. The method of claim 15, wherein the step of performing an intermittent traction therapy includes the user remaining in a single treatment position.

17. The method of claim 13, wherein the external structure is selected from the group consisting of a wall and a door frame.

18. The method of claim 13, further comprising:
adjusting a level of applied traction tension delivered by the traction therapy device.

19. The method of claim 18, wherein adjusting a level of applied traction tension includes rotating an end cap relative to a housing of the traction control unit.
